(19) United States
(12) Patent Application Publication
(10) Pub. No.: US 2022/0175274 A1
(43) Pub. Date: Jun. 9, 2022

(54) INFORMATION PROCESSING DEVICE, STATE DETERMINATION SYSTEM, ENERGY CALCULATION SYSTEM, INFORMATION PROCESSING METHOD, AND STORAGE MEDIUM

(71) Applicant: NEC Corporation, Minato-ku, Tokyo (JP)

(72) Inventors: Chenhui HUANG, Tokyo (JP); Kenichiro FUKUSHl, Tokyo (JP)

(73) Assignee: NEC Corporation, Minato-ku, Tokyo (JP)

(21) Appl. No.: 17/617,398

(22) PCT Filed: Jun. 12, 2019
(86) PCT No.: PCT/JP2019/023360

§ 371 Date: Dec. 8, 2021

(51) Int. Cl. A61B 5/103; A61B 5/11; A61B 5/00

(52) U.S. Cl. CPC A61B 5/1036; A61B 5/1123; A61B 5/4866; A61B 5/6807; A61B 5/7257; A61B 5/1118

(57) ABSTRACT

Provided is an information processing device including an acquisition unit configured to acquire first load information measured by a first load measurement device provided on a sole of a user and second load information measured by a second load measurement device provided to be closer to a toe of the sole than the first load measurement device and a determination unit configured to determine whether or not the user is in a pedaling state in which the user pedals a bicycle based on the first load information and the second load information.

### TECHNICAL FIELD

The present invention relates to an information processing device, a state determination system, an energy calculation system, an information processing method, and a storage medium.

### BACKGROUND ART

Patent Literature 1 discloses a device for determining a pose using an acceleration sensor mounted on a human body. The device of the Patent Literature 1 determines whether the person is walking, running, lying, sitting, or standing based on the three axial acceleration acquired by the acceleration sensor.

### CITATION LIST

### Patent Literature

PTL 1: Japanese Patent Application Laid-open No. 2010-125239

### SUMMARY OF INVENTION

### Technical Problem

The state of the user in daily life includes riding of a bicycle in addition to walking, running, lying, sitting and standing which are subject to determination in Patent Literature 1. However, Patent Literature 1 does not disclose a pose determination method applicable to determination of a state of a user riding a bicycle.

The present invention intends to provide an information processing device, a state determination system, an energy calculation system, an information processing method, and a storage medium which can determine a state of a user riding a bicycle with high accuracy.

### Solution to Problem

According to one example aspect of the invention, provided is an information processing device including an acquisition unit configured to acquire first load information measured by a first load measurement device provided on a sole of a user and second load information measured by a second load measurement device provided to be closer to a toe of the sole than the first load measurement device and a determination unit configured to determine whether or not the user is in a pedaling state in which the user pedals a bicycle based on the first load information and the second load information.

According to another example aspect of the invention, provided is an information processing method including acquiring first load information measured by a first load measurement device provided on a sole of a user and second load information measured by a second load measurement device provided to be closer to a toe of the sole than the first load measurement device and determining whether or not the user is in a pedaling state in which the user pedals a bicycle based on the first load information and the second load information.

According to another example aspect of the invention, provided is a storage medium storing a program that causes a computer to perform acquiring first load information measured by a first load measurement device provided on a sole of a user and second load information measured by a second load measurement device provided to be closer to a toe of the sole than the first load measurement device and determining whether or not the user is in a pedaling state in which the user pedals a bicycle based on the first load information and the second load information.

### ADVANTAGEOUS EFFECTS OF INVENTION

According to the present invention, an information processing device, a state determination system, an energy calculation system, an information processing method, and a storage medium which can properly determine a state of a user riding a bicycle with high accuracy can be provided.

### BRIEF DESCRIPTION OF DRAWINGS

FIG. 1 is a schematic diagram illustrating a general configuration of a state determination system according to a first example embodiment.

FIG. 2 is a schematic diagram illustrating an arrangement of a load measurement device according to the first example embodiment.

FIG. 3 is a block diagram illustrating a hardware configuration of a state determination device according to the first example embodiment.

FIG. 4 is a block diagram illustrating a hardware configuration of an information processing terminal according to the first example embodiment.

FIG. 5 is a functional block diagram of an information processing device according to the first example embodiment.

FIG. 6 is a flowchart illustrating an example of a state determination process performed by the state determination device according to the first example embodiment.

FIG. 7 is a flowchart illustrating an example of a pedaling state determination process.

FIG. 8 is a side view of a foot of a user in a pedaling state.

FIG. 9 is a side view of the foot of the user in a walking state.

FIG. 10 is a side view of the foot of the user in the walking state.

FIG. 11 is a graph illustrating an example of first time series data and second time series data when the user is walking.

FIG. 12 is a graph illustrating an example of a first frequency spectrum and a second frequency spectrum when the user is walking.

FIG. 13 is a graph illustrating an example of first time series data and second time series data in the pedaling state.

FIG. 14 is a graph illustrating an example of a first frequency spectrum and a second frequency spectrum in the pedaling state.

FIG. 15 is a functional block diagram of an information processing device according to a second example embodiment.

FIG. 16 is a flowchart illustrating an example of an energy calculation process performed by the energy calculation unit according to the second example embodiment.

FIG. 17 is a functional block diagram of an information processing device according to a third example embodiment.

Exemplary embodiments of the present invention are described below with reference to the drawings. Throughout the drawings, the same components or corresponding components are labeled with same references, and the description thereof may be omitted or simplified.

### FIRST EXAMPLE EMBODIMENT

A state determination system according to the present example embodiment is described. The state determination system of the present example embodiment is a system for measuring and analyzing a state of a user including determination of the state of the user riding the bicycle. As a part of health management, there is a need to acquire logs related to exercise such as daily walking time, bicycle riding time, and the like. In order to acquire the log of the bicycle riding time of the user, a function of determining the state of the user riding the bicycle is required. Accordingly, the present example embodiment provides a state determination system capable of determining the state of the user riding the bicycle with high accuracy.

A state of the user riding the bicycle typically includes a pedaling state in which the user is pedaling the bicycle. In other words, the state determination system of the present example embodiment can determine whether or not the user is pedaling.

Even when the user 4 is on the bicycle, a state in which the user 4 is not pedaling is not included in the pedaling state. Such a state in which the user 4 is not pedaling is called a non-pedaling state. In recent years, a bicycle which is commercially available has been provided with a freewheel mechanism so that the bicycle can be traveled by inertia without turning a pedal. In such riding of the bicycle, a state in which the bicycle is traveling with inertia without pedaling by the user is included in the non-pedaling state. Further, the non-pedaling state includes a state in which the user is not pedaling in the operation of a bicycle equipped with a motor, which is provided with both a pedal and a motor such as a moped and is capable of traveling with human power.

In this specification, the number of wheels included in the bicycle is not particularly limited, and the “bicycle” may include not only a two-wheel bicycle but also a three-wheel bicycle, a bicycle with an auxiliary wheel, and the like. Further, even a vehicle equipped with a motor such as an electrically assisted bicycle or a bicycle with a motor is included in “bicycle” as long as it is provided with a mechanism capable of being driven by a pedal with human power. Further, the “bicycle” includes a stationary bicycle such as a bicycle for indoor training having a pedal like a two-wheel bicycle.

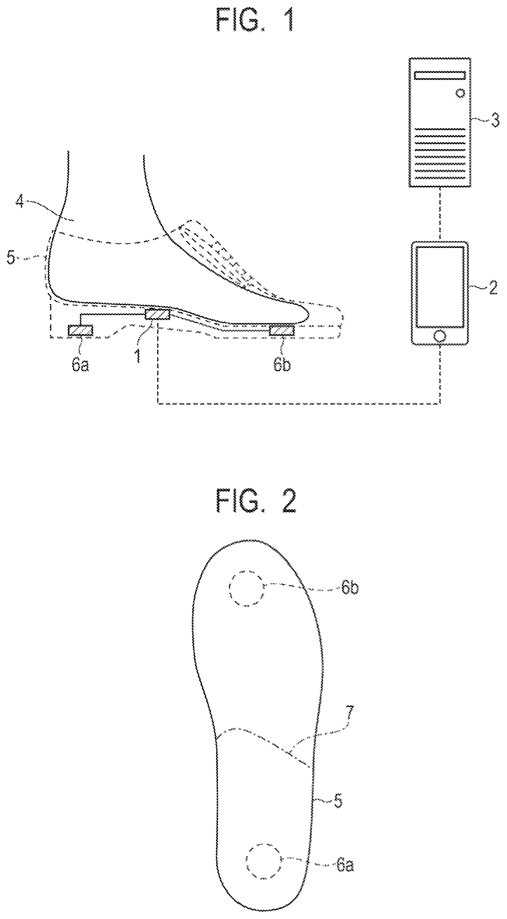

FIG. 1 is a schematic diagram illustrating a general configuration of a state determination system according to the present example embodiment. The state determination system includes a state determination device 1, an information communication terminal 2, a server 3, and load measurement devices 6a and 6b, which can be connected to each other by wireless communication. The load measurement device 6a may be referred to as a first load measurement device, and the load measurement device 6b may be referred to as a second load measurement device.

The state determination device 1 and the load measurement devices 6a and 6b are provided to be close to the sole of a shoe 5 worn by a user 4, for example. The state determination device 1 and the load measurement device 6a, and the state determination device 1 and the load measurement device 6b are communicatively connected by wiring or the like. The load measurement devices 6a and 6b are sensors for measuring load received from the sole of the user 4. The load measurement devices 6a and 6b convert load received from the user 4 into electrical signals and output the electrical signals to the state determination device 1 under the control of the state determination device 1. The load conversion method of the load measurement devices 6a and 6b may be a spring type, a piezoelectric element type, a magnetostrictive type, an electrostatic capacitance type, a gyro type, a strain gauge type, or the like, but is not particularly limited. The load measurement devices 6a and 6b are sometimes referred to as load cells. The state determination device 1 is an electronic apparatus having a control function of the load measurement devices 6a and 6b, an information processing function of analyzing measured load information, a communication function with the information communication terminal 2, or the like.

Note that, the state determination device 1 and load measurement devices 6a and 6b may be provided in the insole of the shoe 5, may be provided in the outsole of the shoe 5, or may be embedded in the shoe 5. The state determination device 1 and the load measurement devices 6a and 6b may be detachably attached to the shoe 5 or may be non-detachably fixed to the shoe 5. The state determination device 1 and the load measurement devices 6a and 6b may be provided at a portion other than the shoe 5 as long as the state determination device 1 can measure the load of the foot. For example, the state determination device 1 may be provided in a sock which the user 4 is wearing, provided in a decoration, directly attached to the foot of the user 4, or embedded in the foot of the user 4. Although FIG. 1 illustrates an example in which one state determination device 1 and two load measurement devices 6a and 6b are provided on one foot of the user 4, one state determination device 1 and two load measurement devices 6a and 6b may be provided on each of both feet of the user 4. In this case, the load information of both feet can be acquired in parallel, and more information can be acquired.

In this specification, the “foot” means a body part below an ankle of the user 4. In addition, in this specification, the “user” means a person who is an object of a determination of a state using the state determination device 1. Whether or not the user corresponds to the “user” is unrelated to whether or not the user is a user of a device other than the state determination device 1 constituting the state determination system, whether or not the user receives a service provided by the state determination system, or the like.

The information communication terminal 2 is a terminal device carried by the user 4, such as a cellular phone, a smartphone, or a smart watch. Application software for analyzing a state is installed in advance in the information communication terminal 2, and processing based on the application software is performed. The information communication terminal 2 acquires data such as the state determination result acquired by the state determination device 1 from the state determination device 1 and performs information processing using the data. The result of the information processing may be notified to the user 4 or may be transmitted to the server 3. The information communication terminal 2 may have a function of providing software such as a control program of the state determination device 1 or a data analysis program to the state determination device 1.

The server 3 provides application software for analyzing states to the information communication terminal 2 and updates the application software. The server 3 may store data acquired from the information communication terminal 2 and perform information processing using the data.

Note that, the general configuration is an example, and for example, the state determination device 1 may be directly connected to the server 3. Further, the state determination device 1 and the information communication terminal 2 may be configured as an integrated device, and another device such as an edge server or a relay device may be further included in the state determination system.

FIG. 2 is a schematic diagram illustrating an arrangement of load measurement devices 6a and 6b according to the present example embodiment. FIG. 2 is a perspective view of the shoe 5 viewed from the bottom side. The load measurement device 6a is provided at a position corresponding to the heel of the user 4, and the load measurement device 6b is provided between the toe and the load measurement device 6a. More specifically, the load measurement device 6a is provided between the position corresponding to the Lisfranc joint 7 of the foot (the joint between the metatarsal bone and the tarsal bone of the foot) and the heel, and the load measurement device 6b is provided between the position corresponding to the Lisfranc joint 7 of the foot and the toe. A dashed dotted line with reference numeral “7” in the figure indicates the position of the Lisfranc joint 7 when the user 4 wears the shoe 5.

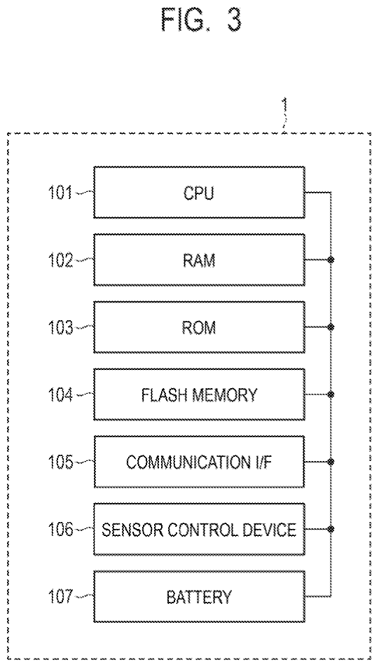

FIG. 3 is a block diagram illustrating a hardware configuration example of the state determination device 1. The state determination device 1 is, for example, a microcomputer or a microcontroller. The state determination device 1 includes a central processing unit (CPU) 101, a random access memory (RAM) 102, a read only memory (ROM) 103, a flash memory 104, a communication interface (I/F) 105, a sensor control device 106, and a battery 107. Each unit in the state determination device 1 is connected each other via a bus, wiring, a driving device, or the like.

The CPU 101 is a processor that performs predetermined calculation in accordance with a program stored in the ROM 103, the flash memory 104, or the like, and also has a function of controlling each unit of the state determination device 1. The RAM 102 is composed of a volatile storage medium and provides a temporary memory area required for the operation of the CPU 101. The ROM 103 is composed of a non-volatile storage medium and stores necessary information such as a program used for the operation of the state determination device 1. The flash memory 104 is a storage device composed of a non-volatile storage medium and temporarily storing data, storing an operation program of the state determination device 1, or the like.

The communication I/F 105 is a communication interface based on standards such as Bluetooth (registered trademark) and Wi-Fi (registered trademark), and is a module for performing communication with the information communication terminal 2.

The sensor control device 106 is a control device that controls the load measurement devices 6a and 6b to measure load and acquires an electric signal indicating the load from the load measurement devices 6a and 6b. The acquired electrical signal is stored in the flash memory 104 as digital data. Thus, the state determination device 1 can acquire the load measured by the load measurement devices 6a and 6b as time series data. Note that, in the present example embodiment, the interval between the data points of the acquired time series data may or may not be constant. The load measured by the load measurement device 6a may be referred to as first load information, and the load measured by the load measurement device 6b may be referred to as second load information. The time series data of the load measured by the load measurement device 6a may be referred to as first time series data, and the time series data of the load measured by the load measurement device 6b may be referred to as second time series data. Note that analog-to-digital (AD) conversion for converting analog signals measured by the load measurement devices 6a and 6b into digital data may be performed in the load measurement devices 6a and 6b, or may be performed by the sensor control device 106.

The battery 107 is, for example, a secondary battery, and supplies power necessary for the operations of the state determination device 1. When power is required to be supplied to the load measurement devices 6a and 6b, the battery 107 may also supply power to the load measurement devices 6a and 6b. Since the battery 107 is built in the state determination device 1, the state determination device can operate wirelessly without connecting to an external power source by wire.

Note that the hardware configuration illustrated in FIG. 3 is an example, and other devices may be added or some devices may not be provided. Further, some devices may be replaced by other devices having similar functions. For example, the state determination device 1 may further include an input device such as a button so that an operation by the user 4 can be accepted, and may further include an output device such as a display, a display lamp, and a speaker for providing information to the user 4. Thus, the hardware configuration illustrated in FIG. 3 can be changed appropriately.

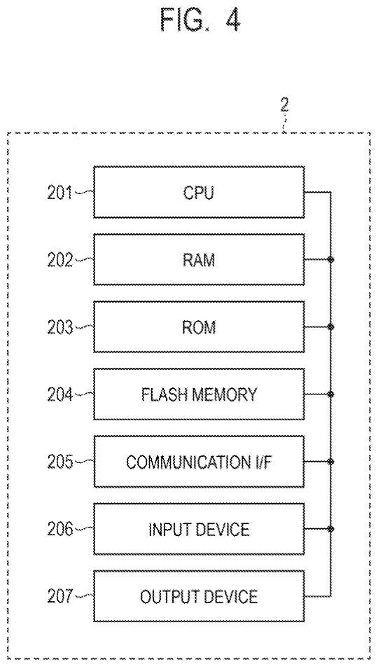

FIG. 4 is a block diagram illustrating a hardware configuration example of the information communication terminal 2. The information communication terminal 2 includes a CPU 201, a RAM 202, a ROM 203, and a flash memory 204. The information communication terminal 2 also includes a communication I/F 205, an input device 206, and an output device 207. Each unit of the information communication terminal 2 is connected to each other via a bus, wiring, a driving device, or the like.

In FIG. 4, each unit constituting the information communication terminal 2 is illustrated as an integrated device, but some of these functions may be provided by an external device. For example, the input device 206 and the output device 207 may be external devices different from those constituting the functions of the computer including the CPU 201 or the like.

The CPU 201 is a processor that performs predetermined calculation in accordance with a program stored in the ROM 203, the flash memory 204, or the like, and also has a function of controlling each unit of the information communication terminal 2. The RAM 202 is composed of a volatile storage medium and provides a temporary memory area required for the operation of the CPU 201. The ROM 203 is composed of a non-volatile storage medium and stores necessary information such as a program used for the operation of the information communication terminal 2. The flash memory 204 is a storage device composed of a non-volatile storage medium for storing data transmitted and received to and from the state determination device and for storing a program for operating the information communication terminal 2.

The communication I/F 205 is a communication interface based on standards such as Bluetooth (registered trademark), Wi-Fi (registered trademark), or 4G and is a module for performing communication with other devices.

The input device 206 is a user interface used by the user 4 to operate the information communication terminal 2. Examples of the input device 206 include a mouse, a trackball, a touch panel, a pen tablet, a button, or the like.

The output device 207 is, for example, a display device. The display device is a liquid crystal display, an organic light emitting diode (OLED) display, or the like, and is used for displaying information, displaying a graphical user interface (GUI) for operation input, or the like. The input device 206 and the output device 207 may be integrally formed as a touch panel.

Note that the hardware configuration illustrated in FIG. 4 is an example, and other devices may be added or some devices may not be provided. Further, some devices may be replaced by other devices having similar functions. Further, some functions of the present example embodiment may be provided by other devices via a network, or some functions of the present example embodiment may be realized by being distributed among a plurality of devices. For example, the flash memory 204 may be replaced by a hard disk drive (HDD) or a cloud storage. Thus, the hardware configuration illustrated in FIG. 4 can be changed appropriately.

The server 3 is a computer having substantially the same hardware configuration as that illustrated in FIG. 4. Since the hardware configuration of the server 3 is substantially the same as that of the information communication terminal 2 except that the server 3 may not be portable, a detailed description thereof is omitted.

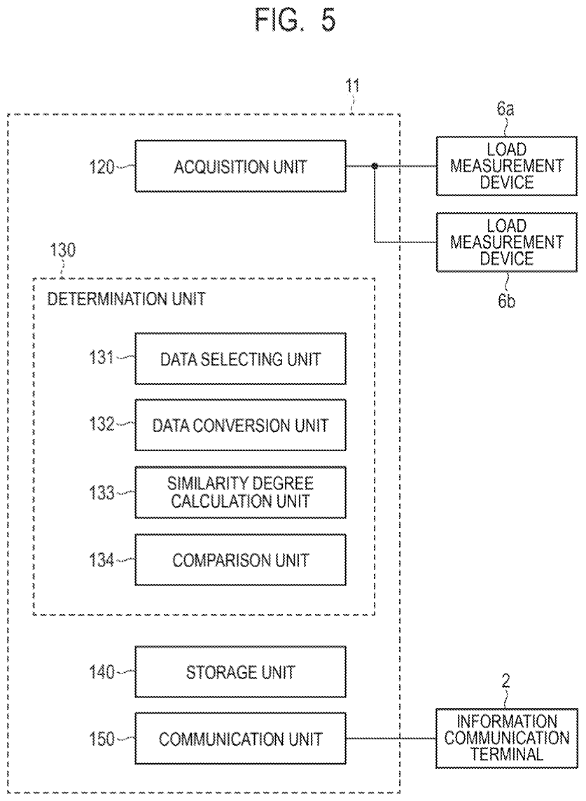

FIG. 5 is a functional block diagram of the information processing device 11 according to the present example embodiment. The information processing device 11 is a portion responsible for an information processing function in the state determination device 1, and a portion of the state determination device 1 may correspond to the information processing device 11, or the entire state determination device 1 may correspond to the information processing device 11. The information processing device 11 includes an acquisition unit 120, a determination unit 130, a storage unit 140, and a communication unit 150. The determination unit 130 includes a data selecting unit 131, a data conversion unit 132, a similarity degree calculation unit 133, and a comparison unit 134.

The CPU 101 loads a program stored in the ROM 103, the flash memory 104, or the like into the RAM 102 and executes the program. Thus, the CPU 101 realizes the functions of the determination unit 130. Further, the CPU 101 realizes the function of the acquisition unit 120 by controlling the sensor control device 106 based on the program. The CPU 101 realizes the function of the storage unit 140 by controlling the flash memory 104 based on the program. Further, the CPU 101 realizes the function of the communication unit 150 by controlling the communication I/F 105 based on the program. Specific processing performed by these units is described later.

In the present example embodiment, each function of the functional blocks illustrated in FIG. 5 is provided in the state determination device 1, but some functions of the functional blocks illustrated in Fig. may be provided in the information communication terminal 2 or the server 3. That is, the above-described functions may be realized by any of the state determination device 1, the information communication terminal 2, and the server 3, or may be realized by cooperation of the state determination device 1, the information communication terminal 2, and the server 3.

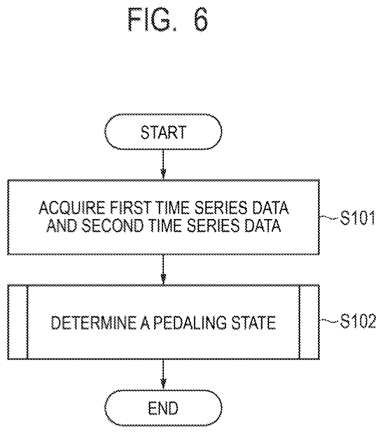

FIG. 6 is a flowchart illustrating an example of a state determination process performed by the state determination device 1 according to the present example embodiment. The process of FIG. 6 is performed at predetermined time intervals, for example. Alternatively, the process of FIG. 6 may be performed when the state determination device 1 detects that the user 4 has got on the bicycle based on a change in load or the like.

In step S101, the acquisition unit 120 controls the load measurement devices 6a and 6b to acquire time series data of load from the load measurement devices 6a and 6b. That is, the acquisition unit 120 acquires the first time series data from the load measurement device 6a and acquires the second time series data from the load measurement device 6b. Thus, the acquisition unit 120 can acquire time changes in the load caused by pedaling of the user 4 or the like. The acquired time series data of the load is converted into digital data and then stored in the storage unit 140. In addition, the time series data of the load is referred to as load information because it indicates time change in load. The load information can be used not only for the state determination of the present example embodiment but also for the weight estimation of the user 4 or personal identification.

Here, in order to sufficiently acquire the feature included in the pedaling, it is desirable that first time series data and second time series data include data in a period corresponding to at least two pedaling cycles (rotation time corresponding to two cycles of the pedal). This is because the pedaling is a substantially periodic circular motion, and therefore, if at least two cycles can be extracted, it can be estimated that the same motion is repeated before and after the two cycles.

In step S102, based on the first time series data and the second time series data, the determination unit 130 performs a pedaling state determination process for determining whether or not the user 4 is in a pedaling state in which the user 4 is pedaling the bicycle.

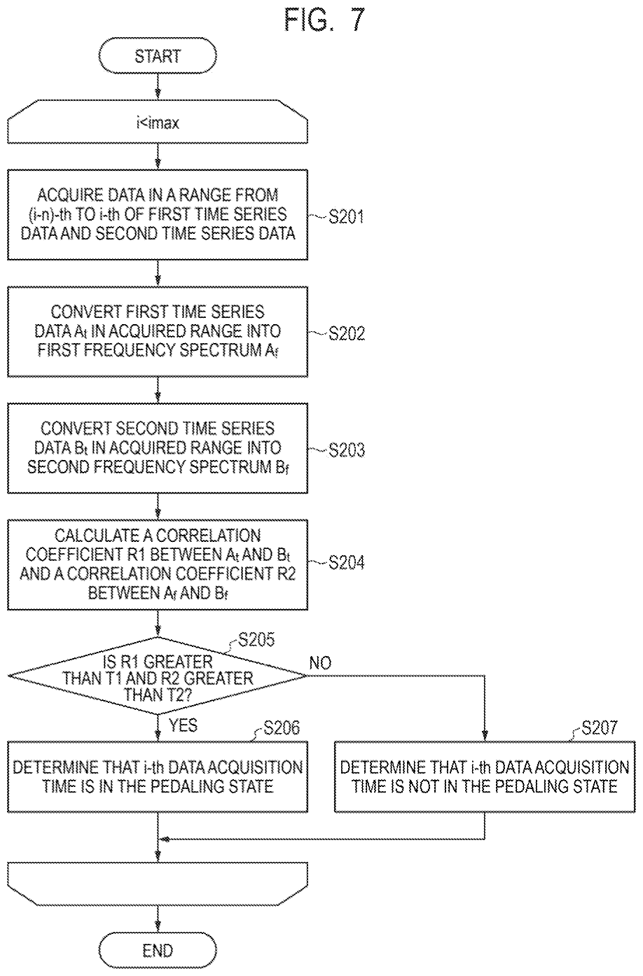

FIG. 7 is a flowchart illustrating an example of a pedaling state determination process. The process of FIG. 7 is a subroutine corresponding to step S102 of FIG. 6. This process is a loop process in which steps S201 through S207 are repeated for each data. In FIG. 7, i represents a data number of time series data of the input first time series data and second time series data. The process steps S201 through S207 are repeated until the data number reaches the predetermined upper limit value imax from the initial value.

In step S201, the data selecting unit 131 acquires data in the range from the (i-n)-th to the i-th of first time series data and second time series data. This process is for specifying a time range of time series data used for conversion into a frequency domain in step S202 and step S203 described later. Therefore, the process of the data selecting unit 131 corresponds to a process of multiplying the time series data by a rectangular window having a width n. Note that the process may be modified to use another window function, and for example, a Gaussian window, a Hanning window, or the like may be applied.

In step S202, the data conversion unit 132 converts the first time series data Aₜ in the range acquired in step S201 into a first frequency spectrum A_{f}. This process may be any process as long as it can convert time domain data into frequency domain data, and may be Fourier transform, for example. The algorithm used for the Fourier transform may be, for example, a fast Fourier transform.

In step S203, as in step S202, the data conversion unit 132 converts the second time series data Bₜ in the range acquired in step S201 into a second frequency spectrum B_{f}.

In step S204, the similarity degree calculation unit 133 calculates a correlation coefficient R1 between the first time series data Aₜ and the second time series data Bₜ. Further, the similarity degree calculation unit 133 calculates a correlation coefficient R2 between the first frequency spectrum A_{f} and the second frequency spectrum B_{f}. Note that the correlation coefficients R1 and R2 may typically be Pearson's product moment correlation coefficients. The correlation coefficients R1 and R2 may be referred to as a first similarity degree and a second similarity degree, respectively.

In step S205, the comparison unit 134 compares the correlation coefficients R1 and R2 with predetermined threshold values T1 and T2. When the correlation coefficient R1 is greater than the threshold value T1 and the correlation coefficient R2 is greater than the threshold value T2 (YES in step S205), the process proceeds to step S206. If the above condition is not satisfied (NO in step S205), the process proceeds to step S207. The threshold values T1 and T2 may be more generally referred to as a first threshold value and a second threshold value, respectively.

In step S206, the determination unit 130 determines that the user 4 pedaled the bicycle at the i-th data acquisition time (that is, the user 4 was in the pedaling state). The determination result is stored in the storage unit 140 in association with the data number i or the time corresponding thereto.

In step S207, the determination unit 130 determines that the user 4 did not pedal the bicycle at the i-th data acquisition time (that is, the user 4 was not in the pedaling state). The determination result is stored in the storage unit 140 in association with the data number i or the time corresponding thereto.

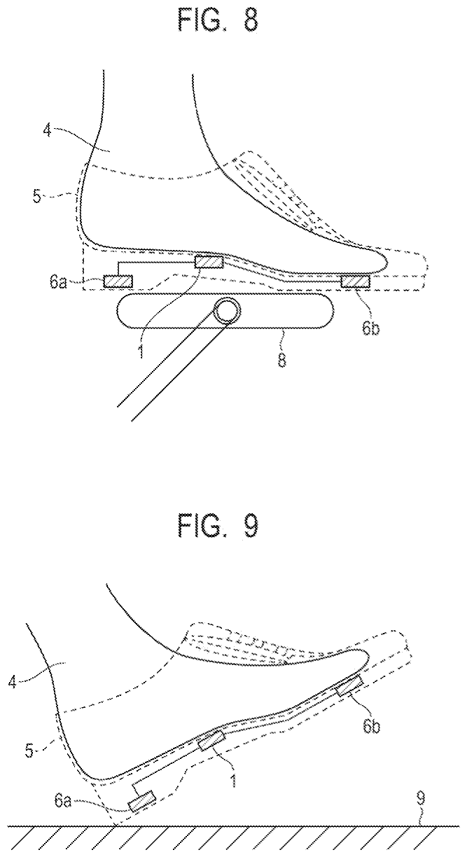

In the above-described pedaling state determination process, two load information acquired from different positions of the sole are used for determination. The reason why it is possible to accurately determine whether or not the user 4 is pedaling is described. FIG. 8 is a side view of the foot of the user 4 in the pedaling state. As illustrated in FIG. 8, at the time of pedaling, the sole of the user 4 is in contact with the pedal 8. When the user 4 turns the pedal 8, the load applied to the pedal 8 from the sole of the foot changes depending on the position of the pedal 8 (the phase of the rotation of the pedal 8). However, when the user 4 turns the pedal 8, a force is applied to the two load measurement devices 6a and 6b at substantially the same time, so that the phases of the loads measured by the two load measurement devices 6a and 6b (peak times of the load) approximately coincide with each other.

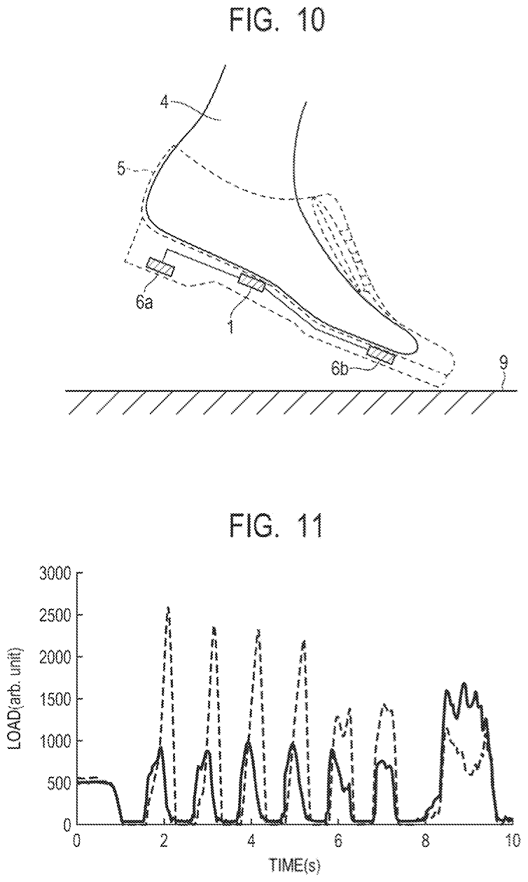

In contrast, in many cases, the phases of load measured by the two load measurement devices 6a and 6b are different from each other in a state other than the pedaling state. A case where the user 4 is walking on level ground is described as an example. FIG. 9 and FIG. 10 are side views of the foot of the user 4 in the walking state. FIG. 9 illustrates the moment when the foot of the user 4 lands on ground 9. When the foot of the user 4 lands on the ground 9, the heel normally contacts the ground 9 first, and then the toe contacts the ground 9. FIG. 10 illustrates the moment when the foot of the user 4 leaves the ground 9. When the foot of the user 4 leaves the ground 9, the heel usually leaves the ground 9 first, and the toe then leaves the ground 9. In this way, during walking on level ground, forces are applied to the two load measurement devices 6a and 6b at different time, so that the phases (peak times of load) of load measured by the two load measurement devices 6a and 6b are different from each other.

Therefore, the determination accuracy can be improved by using the two load information acquired from the two load measurement devices 6a and 6b provided at different positions of the sole for determination of the pedaling state. For the above reason, it is desirable that the two load measurement devices 6a and 6b be provided apart from each other in the front-back direction of the foot. Typically, as illustrated in FIG. 2, it is desirable that the load measurement device 6a be provided between the heel and the Lisfranc joint 7, and the load measurement device 6b be provided between the toe and the Lisfranc joint 7.

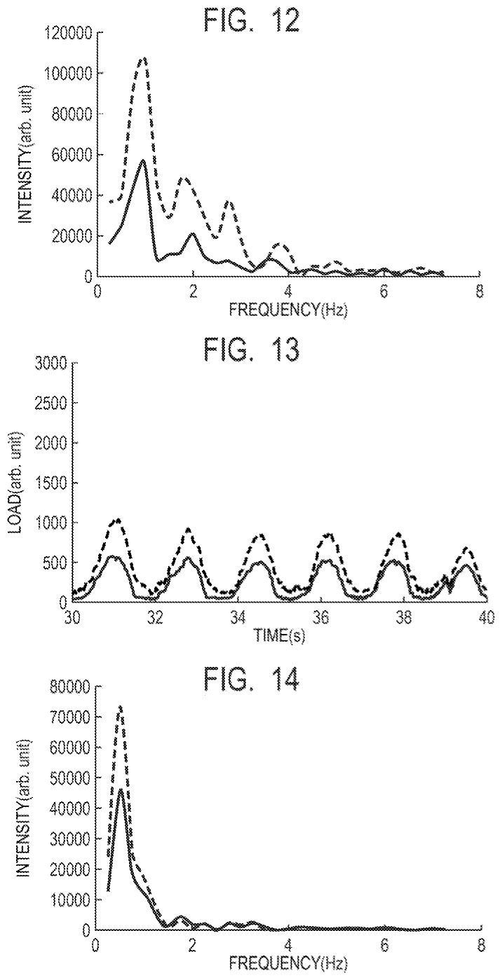

In the above-described pedaling state determination process, determination is performed using correlation coefficients of two data. The reason why whether or not the user 4 is pedaling can be determined with higher accuracy is described. First, a waveform of load when the user 4 is walking is described with reference to FIG. 11 and FIG. 12 as an example of a case where the user 4 is not pedaling (non-pedaling state). FIG. 11 is a graph illustrating an example of the first time series data and the second time series data when the user 4 is walking. The horizontal axis of FIG. 11 represents the time in units of seconds, and the vertical axis of FIG. 11 represents the load in arbitrary units measured by each of the load measurement devices 6a and 6b. The solid line graph of FIG. 11 illustrates the load acquired by the load measurement device 6a, that is, the first time series data, and the broken line graph of FIG. 11 illustrates the load acquired by the load measurement device 6b, that is, the second time series data.

FIG. 12 is a graph illustrating an example of a first frequency spectrum and a second frequency spectrum when the user 4 is walking. The horizontal axis of FIG. 12 represents the frequency in units of Hertz (Hz), and the vertical axis of FIG. 12 represents the intensity in arbitrary units. The solid line graph of FIG. 12 represents the first frequency spectrum, and the broken line graph of FIG. 12 represents the second frequency spectrum.

As can be understood from FIG. 11 and FIG. 12, when the user 4 walks, the waveforms based on load acquired from two load measurement devices 6a and 6b are not similar to each other in both the time series data and the frequency spectrum. Therefore, when the user 4 walks, the correlation coefficient between these waveforms is a small value.

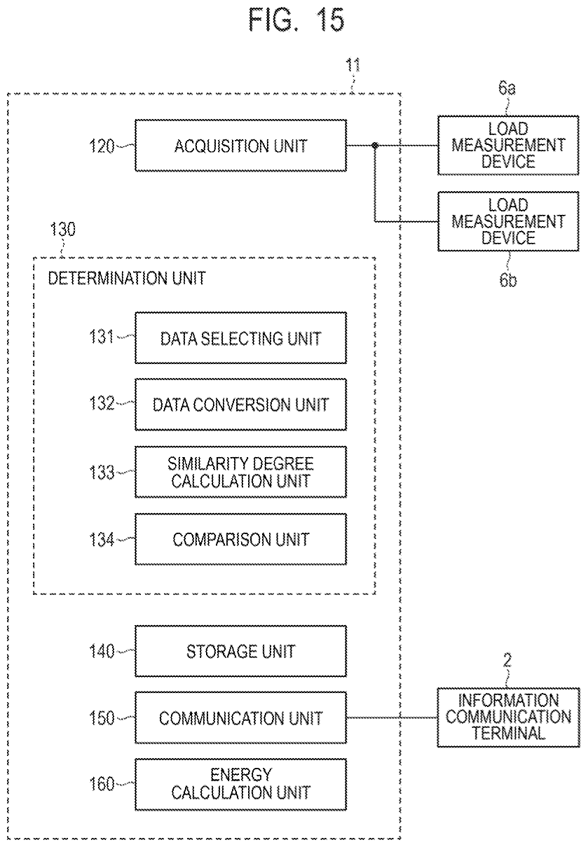

Next, waveforms of load when the user 4 is pedaling (pedaling state) is described with reference to FIG. 13 and FIG. 14. The notations of the graphs are the same as those in FIG. 11 and FIG. 12, and therefore the description thereof is omitted. As can be understood from FIG. 13 and FIG. 14, in the pedaling state, the waveforms based on load acquired from two load measurement devices 6a and 6b are similar to each other in both the time series data and the frequency spectrum. Therefore, in the pedaling state, the correlation coefficient between these waveforms is greater than a case where the user 4 walks.

As described above, in the pedaling state, the similarity degree is high and the correlation coefficient is great as compared with the non-pedaling state. Therefore, the correlation coefficient is calculated as an index of the similarity degree of waveforms, and the magnitude relation between the correlation coefficient and the threshold value is used as the determination condition, whereby it is possible to determine the pedaling state with higher accuracy. An index other than the correlation coefficient may be used as long as the determination method uses the similarity degree of waveforms. For example, covariance may be used as a determination condition.

Further, in this determination, by referring to both the time series data, which is the waveform in the time domain, and the frequency spectrum, which is the waveform in the frequency domain, it is possible to more reliably determine the pedaling state. However, the determination may be performed using only the time series data or using only the frequency spectrum. In this case, the process is simplified, and the amount of calculation can be reduced.

As described above, in the present example embodiment, it is determined whether or not the user 4 is in the pedaling state based on the two load information acquired from the two load measurement devices 6a and 6b provided at different positions of the sole. Thus, the information processing device 11 can accurately determine the state of the user 4 riding the bicycle.

### SECOND EXAMPLE EMBODIMENT

The energy calculation system of the present example embodiment is an example of utilizing the function of determining the pedaling state by the state determination system of the first example embodiment. There is a need to acquire a log of daily energy consumption (so-called consumed calories) as a part of health management. The energy calculation system is a system that can meet the above needs by calculating the energy consumed by the user 4 when the user 4 rides the bicycle. Description of portions common to those in the first example embodiment is omitted.

FIG. 15 is a functional block diagram of the information processing device 11 included in the energy calculation system according to the present example embodiment. The energy calculation system of the present example embodiment is acquired by adding an energy calculation unit 160 to the information processing device 11 of the state determination system of the first example embodiment. The CPU 101 realizes the function of the energy calculation unit 160 by loading a program stored in the ROM 103, the flash memory 104, or the like into the RAM 102 and executing the program. In FIG. 15, the energy calculation unit 160 is provided in the information processing device 11, but this function may be provided in the information communication terminal 2 or the server 3.

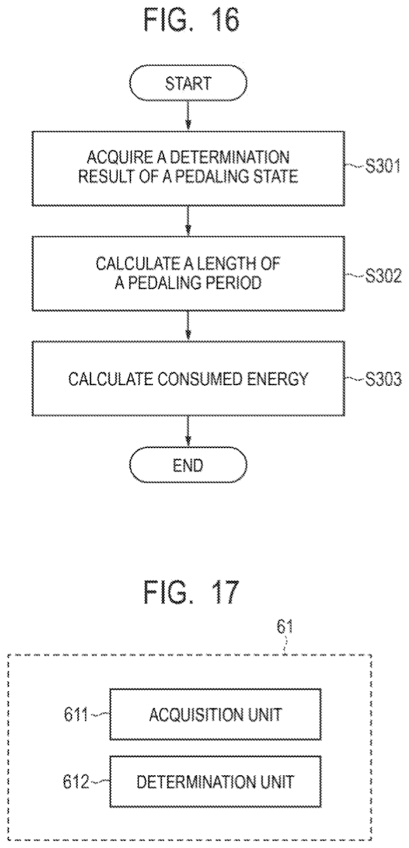

FIG. 16 is a flowchart illustrating an example of an energy calculation process performed by the energy calculation unit 160 according to the present example embodiment. The process of FIG. 16 is performed, for example, after the end of the process according to the flowchart of FIG. 6. Alternatively, the process of FIG. 16 may be performed based on an operation of energy calculation by the user 4.

In step S301, the energy calculation unit 160 acquires the determination result of the pedaling state corresponding to each data acquisition time from the storage unit 140. In step S302, the energy calculation unit 160 adds up the period in the pedaling state (pedaling period) and calculates the length of the pedaling period in the data acquisition period.

In step S303, the energy calculation unit 160 calculates the energy consumed by the user 4 by riding of the bicycle, based on the length of the pedaling period. For example, the following Equation (1) can be used as a calculation equation used for this calculation.

Consumed energy=exercise intensity (METs)×length of pedaling period×body weight×coefficient (1)

In Equation (1), METs, which is a unit of exercise intensity, represents how many times the energy consumption is as compared with the rest state during exercise. Depending on the speed, the inclination of the riding route, and the like, the METs of the bicycle riding is, for example, 4.0 (METs) or 6.8 (METs). The value of the exercise intensity may be input by the user 4 in advance with reference to a METs table or the like, or may be automatically set based on the speed of the bicycle or the like calculated from waveforms of load. In Equation (1), the coefficient is about 1.05 when the unit of the length of the pedaling period is time (hour), the unit of the body weight is kg, and the unit of the consumed energy is kcal.

In the pedaling state, by pedaling, the energy consumption is increased as compared with the case of the non-pedaling state. By focusing attention on the length of the pedaling period, the energy calculation unit 160 of the present example embodiment can calculate the consumed energy more accurately than the case where the consumed energy is calculated based only on the length of time during which the user 4 is on the bicycle.

The energy calculation system of the present example embodiment uses the information processing device 11 that can accurately determine the state of the user 4 riding the bicycle. Thus, an energy calculation system capable of accurately calculating consumed energy is provided.

The device or system described in the above example embodiments can also be configured as in the following third example embodiment.

### THIRD EXAMPLE EMBODIMENT

FIG. 17 is a functional block diagram of the information processing device 61 according to the third example embodiment. The information processing device 61 includes an acquisition unit 611 and a determination unit 612. The acquisition unit 611 acquires first load information measured by a first load measurement device provided on a sole of a user and second load information measured by a second load measurement device provided to be closer to a toe of the sole than the first load measurement device. The determination unit 612 determines whether or not the user is in a pedaling state in which the user pedals a bicycle based on the first load information and the second load information.

According to the present example embodiment, the information processing device 61 capable of accurately determining the state of the user riding the bicycle is provided.

### MODIFIED EXAMPLE EMBODIMENTS

The present invention is not limited to the example embodiments described above, and may be suitably modified within the scope of the present invention. For example, an example in which a part of the configuration of one example embodiment is added to another example embodiment or an example in which a part of the configuration of one example embodiment is replaced with another example embodiment is also an example embodiment of the present invention.

In the above-described example embodiments, two load measurement devices 6a and 6b are used, but sensors other than these may also be used. For example, an angular velocity sensor that measures angular velocity in the three axial directions and an acceleration sensor that measures acceleration in the three directions, or a magnetic sensor that detects geomagnetism by detecting magnetism in three directions to identify an azimuth may be further used. Even in this case, the same processing as the above-described example embodiments can be applied, and the accuracy can be further improved. Further, a global positioning system (GPS) receiver may also be used. In this case, the current position of the bicycle can be acquired, and the log of the position information and the speed information can be acquired.

Although the state determination process is performed inside the state determination device 1 in the above-described example embodiment, this function may be provided in the information communication terminal 2. In this case, the information communication terminal 2 functions as a state determination device.

A processing method in which a program for operating the configuration of the above-described example embodiments are recorded in a storage medium so as to implement the functions of the above-described example embodiments, the program recorded in the storage medium is read as code, and the program is executed in a computer is also included in the scope of each example embodiment. That is, a computer-readable storage medium is also included in the scope of the example embodiments. Further, not only the storage medium in which the above program is recorded, but also the program itself is included in each example embodiment. In addition, one or more components included in the above-described example embodiments may be a circuit such as an application specific integrated circuit (ASIC), a field programmable gate array (FPGA) configured to implement the functions of each component.

As the storage medium, for example, a floppy (registered trademark) disk, a hard disk, an optical disk, a magneto-optical disk, a compact disk (CD)-ROM, a magnetic tape, a nonvolatile memory card, or a ROM can be used. Further, the scope of each example embodiment is not limited to the case where the processing is executed by the program alone recorded in the storage medium, and a case where the processing is executed by operating on an operating system (OS) in cooperation with the functions of other software and extension board is also included in the scope of each example embodiment.

The service realized by the functions of the above-described example embodiments may be provided to the user in the form of a software as a service (SaaS).

It should be noted that the above-described example embodiments are merely examples of embodying the present invention, and the technical scope of the present invention should not be limitedly interpreted by these. That is, the present invention can be implemented in various forms without departing from the technical idea or the main features thereof.

The whole or part of the example embodiments disclosed above can be described as, but not limited to, the following supplementary notes.

### Supplementary Note 1

An information processing device comprising:

an acquisition unit configured to acquire first load information measured by a first load measurement device provided on a sole of a user and second load information measured by a second load measurement device provided to be closer to a toe of the sole than the first load measurement device; and

a determination unit configured to determine whether or not the user is in a pedaling state in which the user pedals a bicycle based on the first load information and the second load information.

### Supplementary Note 2

The information processing device according to supplementary note 1,

wherein the first load information includes first time series data indicating a time change of a load measured by the first load measurement device, and

wherein the second load information includes second time series data indicating a time change of a load measured by the second load measurement device.

### Supplementary Note 3

The information processing device according to supplementary note 2, wherein the determination unit determines whether or not the user is in the pedaling state based on the first time series data and the second time series data.

### Supplementary Note 4

The information processing device according to supplementary note 3, wherein the determination unit determines whether or not the user is in the pedaling state based on a first similarity degree between the first time series data and the second time series data.

### Supplementary Note 5

The information processing device according to supplementary note 4, wherein the first similarity degree includes a correlation coefficient between the first time series data and the second time series data.

### Supplementary Note 6

The information processing device according to any one of supplementary notes 3 to 5, wherein the determination unit determines whether or not the user is in the pedaling state further based on a first frequency spectrum acquired by transforming the first time series data into a frequency domain and a second frequency spectrum acquired by transforming the second time series data into a frequency domain.

### Supplementary Note 7

The information processing device according to supplementary note 6, wherein the determination unit determines whether or not the user is in the pedaling state based on a second similarity degree between the first frequency spectrum and the second frequency spectrum.

### Supplementary Note 8

The information processing device according to supplementary note 7, wherein the second similarity degree includes a correlation coefficient between the first frequency spectrum and the second frequency spectrum.

### Supplementary Note 9

The information processing device according to supplementary note 7 or 8, wherein the determination unit determines the user is in the pedaling state in a case where a first similarity degree between the first time series data and the second time series data is greater than a first threshold value and a second similarity degree between the first frequency spectrum and the second frequency spectrum is greater than a second threshold value.

### Supplementary Note 10

The information processing device according to any one of supplementary notes 2 to 9, wherein each of the first time series data and the second time series data includes at least two periods of pedaling cycles.

### Supplementary Note 11

The information processing device according to any one of supplementary notes 1 to 10,

wherein the first load measurement device is provided to be closer to a heel than a Lisfranc joint of a foot of the user, and

wherein the second load measurement device is provided to be closer to a toe than the Lisfranc joint.

### Supplementary Note 12

A state determination system comprising:

the information processing device according to any one of supplementary notes 1 to 11;

the first load measurement device; and

the second load measurement device.

### Supplementary Note 13

An energy calculation system comprising an energy calculation unit configured to calculate energy consumed by the user by riding the bicycle based on a time of the pedaling state acquired by the information processing device according to any one of supplementary notes 1 to 11.

### Supplementary Note 14

An information processing method comprising:

acquiring first load information measured by a first load measurement device provided on a sole of a user and second load information measured by a second load measurement device provided to be closer to a toe of the sole than the first load measurement device; and

determining whether or not the user is in a pedaling state in which the user pedals a bicycle based on the first load information and the second load information.

### Supplementary Note 15

A storage medium storing a program that causes a computer to perform:

acquiring first load information measured by a first load measurement device provided on a sole of a user and second load information measured by a second load measurement device provided to be closer to a toe of the sole than the first load measurement device; and

determining whether or not the user is in a pedaling state in which the user pedals a bicycle based on the first load information and the second load information.

### REFERENCE SIGNS LIST

1 state determination device

2 information communication terminal

3 server

4 user

5 shoe

6a, 6b load measurement device

7 Lisfranc joint

8 pedal

9 ground

11, 61 information processing device

101, 201 CPU

102, 202 RAM

103, 203 ROM

104, 204 flash memory

105, 205 communication I/F

106 sensor control device

107 battery

120, 611 acquisition unit

130, 612 determination unit

131 data selecting unit

132 data conversion unit

133 similarity degree calculation unit

134 comparison unit

140 storage unit

150 communication unit

160 energy calculation unit

206 input device

207 output device

What is claimed is:

1. An information processing device comprising:
a memory configured to store instructions; and
a processor configured to execute the instructions to:
acquire first load information measured by a first load measurement device provided on a sole of a user and second load information measured by a second load measurement device provided to be closer to a toe of the sole than the first load measurement device; and
determine whether or not the user is in a pedaling state in which the user pedals a bicycle based on the first load information and the second load information.

2. The information processing device according to claim 1,
wherein the first load information includes first time series data indicating a time change of a load measured by the first load measurement device, and
wherein the second load information includes second time series data indicating a time change of a load measured by the second load measurement device.

3. The information processing device according to claim 2, wherein whether or not the user is in the pedaling state is determined based on the first time series data and the second time series data.

4. The information processing device according to claim 3, wherein whether or not the user is in the pedaling state is determined based on a first similarity degree between the first time series data and the second time series data.

5. The information processing device according to claim 4, wherein the first similarity degree includes a correlation coefficient between the first time series data and the second time series data.

6. The information processing device according to claim 3, wherein whether or not the user is in the pedaling state is determined further based on a first frequency spectrum acquired by transforming the first time series data into a frequency domain and a second frequency spectrum acquired by transforming the second time series data into a frequency domain.

7. The information processing device according to claim 6, wherein whether or not the user is in the pedaling state is determined based on a second similarity degree between the first frequency spectrum and the second frequency spectrum.

8. The information processing device according to claim 7, wherein the second similarity degree includes a correlation coefficient between the first frequency spectrum and the second frequency spectrum.

9. The information processing device according to claim 7, wherein the user is determined to be in the pedaling state in a case where a first similarity degree between the first time series data and the second time series data is greater than a first threshold value and a second similarity degree between the first frequency spectrum and the second frequency spectrum is greater than a second threshold value.

10. The information processing device according to claim 2, wherein each of the first time series data and the second time series data includes at least two periods of pedaling cycles.

11. The information processing device according to claim 1,
wherein the first load measurement device is provided to be closer to a heel than a Lisfranc joint of a foot of the user, and
wherein the second load measurement device is provided to be closer to a toe than the Lisfranc joint.

12. A state determination system comprising:
the information processing device according to claim 1;
the first load measurement device; and
the second load measurement device.

13. An energy calculation system comprising:
a memory configured to store instructions; and
a processor configured to execute the instructions to calculate energy consumed by the user by riding the bicycle based on a time of the pedaling state acquired by the information processing device according to claim 1.

14. An information processing method comprising:
acquiring first load information measured by a first load measurement device provided on a sole of a user and second load information measured by a second load measurement device provided to be closer to a toe of the sole than the first load measurement device; and
determining whether or not the user is in a pedaling state in which the user pedals a bicycle based on the first load information and the second load information.

15. A non-transitory storage medium storing a program that causes a computer to perform:
acquiring first load information measured by a first load measurement device provided on a sole of a user and second load information measured by a second load measurement device provided to be closer to a toe of the sole than the first load measurement device; and
determining whether or not the user is in a pedaling state in which the user pedals a bicycle based on the first load information and the second load information.
